(19) United States
(12) Patent Application Publication
(10) Pub. No.: US 2023/0092442 A1
(43) Pub. Date: Mar. 23, 2023

(54) DUAL PORT DRAIN

(71) Applicants: DROPLET BIOSCIENCES, INC., Melrose, MA (US); THE WASHINGTON UNIVERSITY, St. Louis, MO (US)

(72) Inventors: Jose P. Zevallos, St. Louis, MO (US); Aadel Chaudhuri, Chesterfield, MO (US); Stanley N. Lapidus, Golden, CO (US)

(21) Appl. No.: 17/947,881

(22) Filed: Sep. 19, 2022

Related U.S. Application Data
(60) Provisional application No. 63/246,255, filed on Sep. 20, 2021.

(51) Int. Cl. A61M 39/02; A61M 27/00

(52) U.S. Cl. CPC A61M 39/0247; A61M 27/00; A61M 2039/0205; A61B 2010/008

(57) ABSTRACT

The present invention relates to novel surgical drains and associated methods and systems. Specifically, the present invention includes a surgical drain with at least two ports. A first port drains lymphatic fluid from a subject for collection and analysis. The second port functions to introduce a therapeutic into the subject.

### TECHNICAL FIELD

This invention provides methods and devices for diagnosing disease and/or assessing patient treatment options based on biomarkers identified from lymphatic fluid.

### BACKGROUND

Many types of diseases and surgical interventions cause a buildup of fluid that must be drained. This often occurs through the use of a surgical drain inserted into a patient. Existing surgical drains include an implantable, inflow drain section placed into subject's body. This is generally a tube. Typically, the inflow drain section is placed in fluidic communication at the site of fluid accumulation, which is often the site of a surgical treatment. The inflow drain section collects and transfers fluid, which generally includes lymphatic fluid, to an outflow section. The outflow section in turn is often connected to a container into which collected fluid accumulates.

Periodically, a subject with an implantable surgical drain empties the container and disposes of the accumulated fluid. Thus, conventionally surgical drains are single-purpose devices—they remove unwanted fluid from a subject disposal.

### SUMMARY

The present invention relates to novel surgical drains and associated methods and systems. Specifically, the present invention includes a surgical drain with at least two ports. A first port drains lymphatic fluid from a subject for collection and analysis. The second port functions to introduce a therapeutic into the subject.

The present Inventors made the surprising discovery that, lymphatic drainage fluid (e.g., from a surgery or investigative assay) contains detectable biomarkers that are useful for diagnosing pathologies, monitoring disease progression and spread, and assessing treatment efficacy. The disclosed surgical drains provide a novel source of diagnostic biomarkers to assess disease status, therapeutic efficacy and the like. Further, the addition of a port for introducing a therapeutic, the presently disclosed surgical drains include a conduit to provide targeted treatments to a subject without any additional incisions or serious discomfort. Moreover, in certain aspects, the drain remains attached during a course of treatment, and biomarkers detected in the collected fluid are used to assess the ongoing efficacy of treatment. As a subject's condition progresses, as measured in whole or part by biomarkers in the collected fluid, new or adjuvant treatments may be administered via the disclosed surgical drains.

Accordingly, the presently-disclosed surgical drains provide greater functionality and utility to a device that traditionally has been used to dispose of biological waste.

In certain aspects, the present invention provides a dual-port drain, which includes a first port for collection of lymphatic or other drainage fluid and a second port for delivery of a therapeutic, which may be a pharmaceutical composition or may simply be saline or other fluids. The second port may deliver, for example, an immunotherapy compound, an antibiotic or antiviral compound, a cancer therapy, nutrients, saline, coagulant/anti-coagulant, and/or any other therapeutic as required, e.g., to promote healing or in response to biomarkers analyzed in the collected drain fluid.

In certain aspects, the first port collects, for example, cellular material in drain fluid. Cellular material may include a tumor cell or cell-free tumor DNA. The first port may include a suction drain to facilitate removing fluid from a subject. The collected material may be any nucleic acid in any form (e.g., DNA, RNA, miRNA and the like), proteins, and other biological molecules indicative of a pathology.

In certain aspects, the dual-port drain includes an assay device or module for biomarker detection. The first port may include a first valve for sealing the first port, which may stop it from collecting drain fluid and/or prepare it for removal of a fluid reservoir, e.g., to empty it when full or collect a sample for a bioassay. Similarly, the second port may include a second valve for sealing the second port from delivering the therapeutic and/or controlling the rate at which the therapeutic enters the port. In certain aspects, the first valve and/or second valve are reversible. The first valve and/or second valve may act independently.

Certain dual-port drains of the invention are configured such that when delivery of the therapeutic is finished, the second valve closes the second port and the first valve closes the first port. Alternatively, certain dual-port drains are configured such that when delivery of the therapeutic is finished, the first valve opens to reinitiate fluid draining. The dual-port valve system may be automated or operated manually.

In certain aspects, the first port, or a device attached thereto, includes a sample filter for capturing solid particles. In certain aspects, the first port, or device attached thereto, further includes a heat source for moderating the temperature of collected lymphatic fluid.

### BRIEF DESCRIPTION OF THE DRAWINGS

FIG. 1 provides a schematic of an exemplary dual-port surgical drain of the invention.

FIG. 2 provides a schematic of an exemplary dual-port surgical drain of the invention.

### DETAILED DESCRIPTION OF THE INVENTION

The present invention relates to novel surgical and/or wound drains and associated methods and systems. Specifically, the present invention includes a drain comprising at least two ports. A first port drains fluid from a collection site in situ analysis. The second port functions to introduce a therapeutic.

The present Inventors made the surprising discovery that, otherwise-discarded fluid (e.g., drain fluid from a surgery or biopsy) drained from a subject contains detectable biomarkers that are useful for diagnosing pathologies, monitoring disease progression and spread, therapeutic selection, and assessing treatment efficacy. Thus, the disclosed surgical drains provide a novel source of biomarkers to assess a subject's condition, which imposes no additional burden or pain on the subject. In addition, a dual-port drain system of the invention provides a means for introducing a therapeutic. Accordingly, the presently disclosed surgical drains provide greater functionality and utility to a device that has otherwise been relegated as part of a path to dispose biological waste.

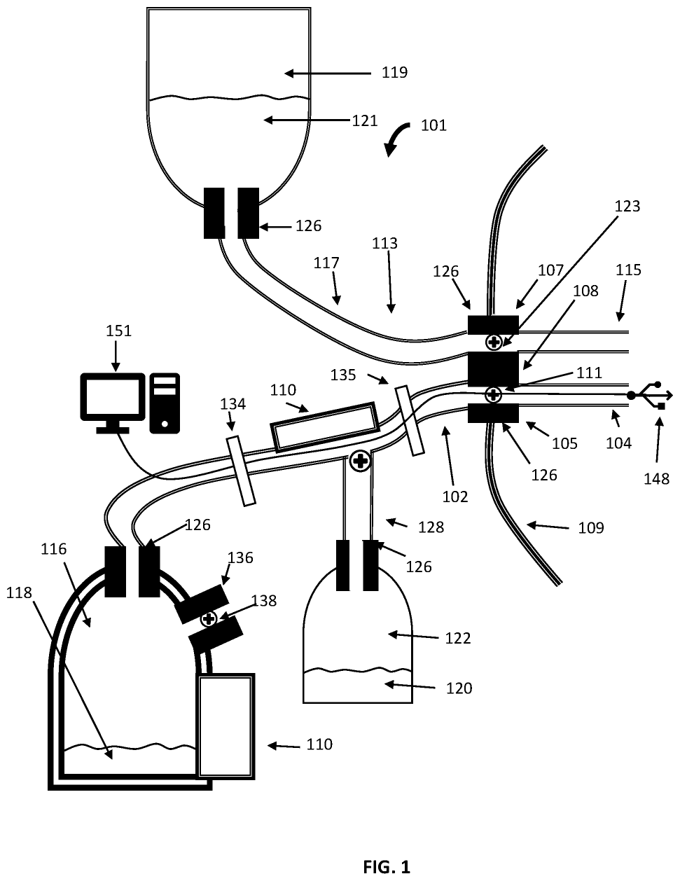

FIG. 1 is a schematic illustration of an exemplary dual-port surgical drain system 101 of the invention. The system 101 includes a skin interface module 108, which includes a first port 105 and second port 107. The skin interface module 108 as affixed on or within the skin 109 of a subject, preferably near a surgical bed, to allow draining. The system includes a surgical drain tube 102, which includes a proximal portion 104 that is configured for implantation within a subject to drain fluid, e.g., from the site of a surgery or fluid buildup.

The drain tube 102 passes through the first port 105, and distal portion 106 of the tube 102 remains external of the subject. In certain aspects, the proximal portion 104 and distal portion 106 of the tube are separate tubes that are fluidically connected via the first port 105 of the skin interface 108. The first port is configured to form a sealed passage through the subject's skin 109 for the surgical drain tube 102. The distal portion 106 of the drain tube 102 extends from first port 105 to a main reservoir 116 that collects fluid 118, which may include lymph fluid, drained from the subject.

In certain aspects, the proximal portion 104 branches into a series of sub-tubes, which can be dispersed, for example, across a wider area within a subject, concurrently into different regions within a subject, and/or into various vascular structures, such as lymphatic vessels and ducts. Thus, using a single insertion point, the systems of the invention can evenly drain fluid from a number of desired locations within a patient. Moreover, if a single branch or sub-tube of the proximal portion becomes blocked or obstructed, e.g., from solid debris in the drain fluid, the system may still function to drain fluid from the subject.

The system 101 further includes a treatment tube 113. The treatment tube 113 includes a distal end 117 that may be in fluidic communication with one or more treatment reservoirs 119, which include a therapeutic 121 for delivery into a subject. The treatment reservoir may be or include, for example, an intravenous bag, a syringe, or a syringe pump. In certain aspects, the therapeutic tube 113 is in fluidic communication with a reservoir that contains saline or another fluid used to irrigate a site within the subject or otherwise clear blockages from the system. The treatment tube 113 includes a proximal portion 115 inserted into a subject via the second port 107. In certain aspects, the proximal portion 115 and distal portion 117 of the tube 113 are separate tubes that are fluidically connected via the second port 107 of the skin interface 108. The second port is configured to form a sealed passage through the subject's skin 109 through which the treatment tube 113, passes, such that a therapeutic 121 can flow through the tube 113 into a subject.

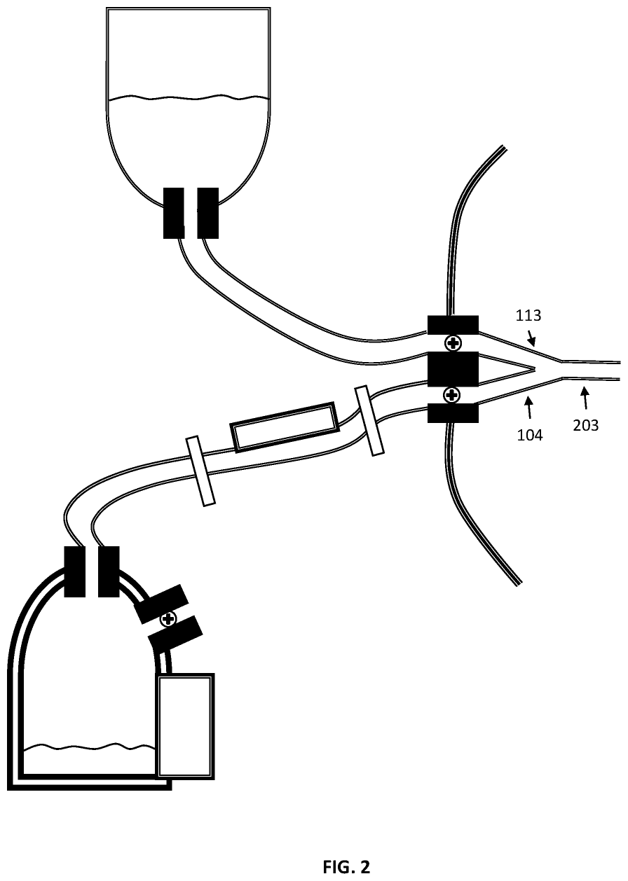

As shown in FIG. 2, in certain aspects, the proximal portion 104 of the drain tube and the proximal portion 115 of the treatment tube are fluidically connected 203. Thus, the system may be used to drain fluid from a particular area in a patient and, using the same tube inserted within the patient, deliver a therapeutic to the drained areas.

In certain aspects, the system 101 includes one or more sample ports 128. As shown in FIG. 1, the sample ports can be in fluidic communication with the sample tube 102. The sample port 128 is configured to divert a portion, or certain components, of the fluid drained using sample tube 102. Alternatively or additionally, a sample port may be in fluidic communication with a reservoir, such as the main reservoir 116. A sample port 128 may be in fluidic communication with a sample reservoir 122 to collect a fluid sample 120 from the drained fluid. Sample reservoirs may be configured to attach and detach from the corresponding sample ports to provide for removal and replacement, such as during sample collection. For example, in the exemplary system 101 in FIG. 1, the sample reservoir 122 may be removed from the sample port 128 when a sufficient fluid sample 120 has been collected. Then, the reservoir 122 may be replaced with an empty reservoir.

In preferred aspects, the system 101 includes a vacuum port 136 with a vacuum valve 138. As shown in FIG. 1, the vacuum port 136 may be associated with the main reservoir 116 and configured to remove air from the main reservoir 116 to create and control a vacuum pressure within the main reservoir 116. The vacuum pressure draws the surgical fluid 118 from the subject into the main reservoir 116 via the surgical drain tube 102. Alternatively, the system 101 may include a vacuum port coupled with the surgical drain tube or first port to provide vacuum pressure to actively drain the fluid through the drain tube.

In certain aspects, a vacuum source is operatively coupled to the vacuum port to create and maintain a vacuum pressure within the main reservoir and/or surgical tube, depending on the configuration. Exemplary vacuum sources include, for example, a suction line, a vacuum tank, a vacuum pump, and any other suitable vacuum source.

Additionally or alternatively, in certain systems of the invention, the fluid is drained, in whole or part, by passively flowing from within the subject to the main reservoir. For example, the flow of the drained fluid may be drawn by gravity from within the subject to the main reservoir, which is preferably located below the first port.

In certain aspects, the system includes a positive pressure port associated with one or more of the treatment tube 113 and treatment reservoir 119. The positive pressure port may provide positive pressure to move a therapeutic 121 from the treatment reservoir, through the treatment tube 113 and second port 107 into the subject. Additionally or alternatively, vacuum pressure associated with the drain tube 102 pulls the therapeutic into the treatment tube and the subject. Additionally or alternatively, in certain systems of the invention, the therapeutic is passively flowed from the treatment reservoir 119 into the subject. For example, the flow therapeutic may be drawn by gravity from treatment reservoir into the treatment tube 113, through the second port 107, and into the subject.

In certain aspects, the treatment reservoir 119 provides positive pressure, e.g., when the reservoir is a syringe, depressing the syringe will cause positive pressure in the system. Alternatively or additionally, the treatment reservoir and/or treatment tube includes one or more positive pressure ports coupled to a positive pressure pump.

The second port 107 may include a valve 123 to regulate or start/stop the flow of therapeutic into the subject. In certain aspects, the valve 123 in the second port cannot be opened while the valve 111 in the first port is open. This may assure, for example, that therapeutic sent into a subject is not immediately drained via the drain tube.

In various other aspects, the main reservoir 116, treatment reservoir(s) 119, the sample port(s) 128, vacuum port(s) 136, positive pressure ports, the first port 105 and/or second port 107 further include reversibly sealable connection fittings 126 configured to interlock and seal with corresponding features throughout the system to form sealed and reversible connections. Any suitable medical-grade connection fitting may be used as a reversibly sealable connection fitting including, for example, Luerlock connectors.

In various aspects, valves of the system 101, such as the valve 138 shown attached to the main reservoir 116, may be opened and closed using any suitable means including, but not limited to, manual opening and closing, actuated opening and closing, and any other suitable means of modulating the position of the valve. In some aspects, a vacuum valve, such as valve 138 is opened to allow the vacuum source to create and/or maintain the vacuum pressure within the main reservoir 116, which causes the system to suck fluid from within the patient through the drain tube via the first port. The valve 138 may be closed to stop an increase in vacuum pressure, once the system achieves a desired negative pressure, when negative pressure is no longer required, and/or when if the vacuum source is inactivated or removed from the vacuum port. Valves (111/123) associated with the first and second ports may be used to independently or collaboratively control the flow of a therapeutic into a subject and fluid drained from the subject.

In certain aspects, in addition or instead of a valve at the main reservoir, the system may include a valve 111 in the first drain port 105. In certain aspects, the system includes a number of valves (e.g., 138/111) associated with the first port and drain line, which work in tandem to control vacuum pressure throughout the system. In certain aspects, the valves (123/111) in the first and second ports may provide a seal when closed to prevent external access through a subject's skin. This allows, for example, the treatment and drain tubes to be removed or replaced without exposing a subject to a risk of contamination through the skin interface. In other aspects, the position of a positive pressure valve or vacuum valve (e.g., valve 138) may be modulated to one or more positions between fully opened and fully closed to modulate the level of pressure created and/or maintained within the system or portions of the system, e.g., the main reservoir 116. In additional aspects, the operation of a vacuum source or positive pressure source may be modulated as needed to control pressure levels in the system or portions thereof.

In some aspects, the vacuum pressure within the main reservoir 116/treatment tube 102 and/or positive pressure in the treatment reservoir 119/treatment tube may be created manually. For example, at least a portion of a reservoir may be constructed using a flexible or elastic material. The material may be compressed (e.g., by hand or via a controlled mechanism) to displace air or fluid out of the reservoir through, thereby creating positive/negative pressure as desired. Once a desired reservoir pressure is obtained, a valve in connection with the reservoir, such as vacuum valve 138, may be closed. In certain aspects, releasing a compressive force from the reservoir causes an elastic rebound of the flexible or elastic materials of the reservoir. This returns the reservoir to its original uncompressed volume, thereby creating a vacuum pressure. In some aspects, a vacuum valve, such as valve 138, may be a one-way valve that permits flow out of the reservoir and prevents backflow of air back into the reservoir, obviating the need manually open/close the vacuum valve during such a pressurization operation.

In certain aspects, the valves (111/123) of the first and second ports (105/107) are one-way backflow prevention valves. The valves permit fluid to drain through tube 102 out of the subject and therapeutic to enter the subject via the treatment tube 113. Such one-way valves may prevent backflow from the drain tube 102 back into the subject and/or fluid from the subject entering the therapeutic tube 113. The backflow prevention valves may be positioned at any suitable position between the first and second ports and the main reservoir 116/treatment reservoir 119. In some aspects, a backflow prevention valve(s) is positioned relatively near the first and/or second ports to assure an appropriate one-way flow through.

In some aspects, the disclosed dual-port drain systems of the invention incorporate modules and/or components that monitor fluid drained from the subject. For example, systems of the invention may incorporate modules and/or components that monitor biomarkers in the drained fluid indicative of a subject's condition or recovery. Biomarkers may provide information regarding a post-operative complication or condition, such as an undermined surgical bed, infections, fistula formation, treatment progress, metabolite or medication concentration, chyle leakage, inflammation, internal bleeding, allergic reactions, immune responses, leaks from areas near a surgical site or repair, and rejection of transplanted cells, organs or tissues. Advantageously, because the systems and devices include a second port, therapeutics or adjuvant treatments may be provided, including as suggested by drain fluid monitoring, quickly and without any need to expose a subject to additional injections or discomfort.

Moreover, in certain instances, the monitored, drained fluid provides an indication about a complication at the drain site, which is often proximal to a surgical bed, injury or infection. Thus, in certain aspects, the treatment tube may provide a needed therapeutic localized to the same area afflicted by an abnormal condition. Similarly, the monitoring may be used to determine the necessary flow rate of therapeutic into the subject based on an assessment of biomarkers in the drain fluid. This may, for example, provide information regarding the therapeutic's efficacy, absorption, and levels in a subject. The rate of flow may be adjusted based on this information.

Systems and devices of the invention may incorporate one or more assay devices to monitor fluid drained from a subject. As shown in FIG. 1, the system includes an assay device 110 coupled to the drain tube 102. An assay device 110 may be operatively coupled to the surgical drain tube 102 at any position along the distal portion 106. In some aspects, the assay device 110 is operatively coupled to the distal portion 106 near the skin interface 108. In certain aspects, the system 101 includes an assay device 112 operatively coupled to the main reservoir 112. In some aspects, an assay device may be, or include, a replaceable cartridge to provide for repeated monitoring of the drain fluids over an extended time period.

Exemplary assay devices include devices capable of detecting biomarkers, such as proteins and other analytes of interest associated with a particular condition and/or post-operative conditions for which the subject is at risk. Suitable assay devices include, for example, immunoassays such as lateral flow immunochromatographic assay devices.

Referring again to FIG. 1, the dual-port drain system 101 may include features to condition the fluid, or components thereof, as it passes through the drain tube 102. For example, the system may include at least one filter 134/135. Preferably, filters are in fluidic communication with the distal portion 106 of the drain tube 102. Filters may be configured to remove one or more components from the drained fluid including, but not limited to, whole cells, clots, and any other component that may potentially interfere with the operation of the assay devices and/or subsequent processing and analysis of the drained fluid. The filters may facilitate the collection, centrifuging, and removal of necrotic debris from the drainage fluid samples.

In certain aspects, the system includes a series of filters or filters fitted before or after sample ports 128. As shown in FIG. 1, the system may include a filter 135 upstream of a sample port 128 and a filter 134 after the port. Thus, certain components or biomarkers may be filtered before entry into the sample port 128. Further components and biomarkers may be filtered out before entry into the main reservoir. In this way the fluid collected in the sample reservoir 120 and main reservoir 116 may include different components for downstream analysis.

In certain aspects, one or more elements of the dual-port drain systems disclosed herein include a coating on surfaces that contact the drained fluid. Exemplary coatings prevent or inhibit the degradation biomarkers of interest, e.g., proteins and nucleic acids, within the drained fluid. The coating(s) may, for example, reduce or prevent denaturing of proteins and/or nucleotides, coagulation, and any other type of degradation of desired biomarkers. Suitable coatings may include EDTA, heparin, and/or trisodium citrate (TSC).

In certain aspects, the systems of the invention include an EDTA coating over at least a portion of the inner surface of the distal portion 106 of the drainage tube 102. In certain aspects, the coating is not included on the proximal portion 104 or the first port 105, because anticoagulant and/or anti-denaturing coating may disrupt healing. In certain aspects, the main reservoir 116 and/or sample reservoir include a contain or different coating to account for biomarkers collected in each reservoir.

The coatings assist the ability of the disclosed systems to collect drainage fluid samples containing intact analytes for downstream analysis. For example, the drainage fluid may be analyzed to detect non-cellular RNA or DNA within a drained fluid sample. Thus, the system may incorporate a coating to inhibit DNA/RNA in the sample from denaturing, thereby enhancing the efficacy of downstream analysis.

In certain aspects, the systems disclosed herein include additional features or components to facilitate collecting high-quality samples of drained fluid. For example, the inner surfaces of the sample port(s) 128 and sample reservoirs 122 may be coated with composition to inhibit denaturing or coagulation of analytes within the fluid samples 120. Non-limiting examples of suitable coating compositions include EDTA, heparin, trisodium citrate (TSC) and any other suitable coating compound, and any combination thereof. In other aspects, elements or components of the system include one or more agents designed to preserve tumor-associated exosomes within the surgical drain tube or reservoirs.

Similarly, the devices may include components configured to preserve the integrity of the biomarker(s) source, e.g., extracellular vesicles and cancer cells. The devices may include components used to perform at least a portion of sample preparation steps, and/or any other suitable function related to obtaining, preserving, and processing the fluid sample.

Examples of suitable biomarkers or fluid components that may be collected using the surgical drain system include circulating tumor cells, exosomes, and cell-free DNA (cfDNA) and RNA. In various aspects, the cell-free DNA and RNA may be associated with a variety of sources including, but not limited to, tumors, solid transplant organs, bacteria, fungi, and viruses, including those near a surgical site where the drain is inserted.

In certain aspects, the analyzed biomarker(s) may include, for example, a biomarker associated with cancer, such as a tumor cell or cell-free tumor DNA. Detecting or quantifying the presence of a cancer biomarker may include sequencing DNA or RNA from a tumor cell or from cell-free tumor DNA, obtained from the fluid.

In certain aspects, the biomarker(s) analyzed in fluid collected from a subject include, for example, tumor cells, immune cells, bacterial cells, viral host cells, donor organ cells, microvascular cells, cell-free DNA (cfDNA), cell-free RNA (cfRNA), circulating tumor DNA (ctDNA), messenger RNA, exosomes, proteins, hormones, and analytes. The biomarker(s) analyzed depend on, for example, a specific patient, pathology, surgery type, and surgery site. By analyzing biomarkers in the obtained fluid, methods of the invention may provide diagnostic or prognostic information. The biomarker(s) may be isolated using methods suitable to the analyte of interest, for example, filtering, and centrifuging, chromatography. In certain aspects, the biomarker includes any one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation. In some embodiments, the biomarker is a ratio of circulating tumor cells to cell-free DNA detected in the fluid.

Systems of the invention may further include features to maintain the drainage fluid at a suitable temperature within, for example, the drainage tube 102, the main reservoir, and/or the sample reservoir(s). This may facilitate preserving intact biomarkers in the collected drained fluid. For example, nucleic acids within the drainage fluid may degrade if the drainage fluid falls outside a suitable temperature range. Thus, systems of the invention may include heating and/or cooling elements configured to maintain or adjust the temperature of certain components of the system to maintain a desired temperature range suitable for maintaining the integrity of DNA and RNA molecules within the drained fluid. Systems of the invention may also or alternatively include thermal insulation positioned around one or more components to facilitate maintenance of an appropriate temperature.

Non-limiting examples of suitable temperature control devices include resistive heaters, piezoelectric heaters, water jacket heaters, and any other suitable heating device.

In other additional aspects, the surgical drain system may further include features to monitor various physiological parameters of the subject or aspects of the site into which the dual-port drain is inserted. For example, the systems may include one or more systems or sensors for monitoring temperature, pH, pressure, hydration, oxygenation, any other suitable physiological parameter, and any combination thereof.

As shown in FIG. 1, the system may include a physiological sensor 148 positioned at the proximal end 104 of the drainage tube 102. Alternatively or additionally, a sensor may be placed at the treatment tube 113, the skin interface 108, the first port 105, and/or the second port 107. The at least one physiological sensor 148 may be configured to monitor at least one physiological parameter within the subject. In various aspects, the at least one physiological sensor 148 may be provided as a single sensor incorporating at least one probes to monitor the physiological parameter, or the at least one physiological sensor may be provided in the form of a separate probe for monitoring each physiological parameter within the surgical bed. As shown, sensors may be inserted into the subject and connected to an external computing system 151 to control and monitor the sensors.

The sensors may be connected to the external computing system 151 that controls and monitors the sensor. Non-limiting suitable communication devices or protocols include connecting cables, wireless communication such as Bluetooth communication protocol, and any other suitable communication device or protocol. In certain aspects, the sensor connects to the computing system 151 through the treatment tube 113, the drain tube 102, the first port 105, and/or the second port 107.

In certain aspects, the computing system 151 or another computing system controls one or more additional aspects of the dual-port drain system. For example, the computing device may regulate one or more valve(s), positive pressure pump, vacuum pump, and/or other components to regulate the flow of drain fluid and/or therapeutic in the system. The computing device may control one or more assay devices or temperature control devices as described herein.

The computing system may include at least one computer, which many include a server computer. The computing system may include a processor coupled to a tangible, non-transitory memory device and at least one input/output device. Thus, components of the system may be in communication over a network that may be wired or wireless and wherein the components may be remotely located or located in close proximity to each other.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as a NVidia Tesla K80 graphics card from NVIDIA Corporation (Santa Clara, Calif.). A processor may be provided by a chip from Intel or AMD. A processor may be any suitable processor such as the microprocessor sold under the trademark XEON ES-2620 v3 by Intel (Santa Clara, Calif.) or the microprocessor sold under the trademark OPTERON 6200 by AMD (Sunnyvale, Calif.). Computer systems of the invention may include multiple processors including CPUs and or GPUs that may perform different functions required by the dual-port drain systems of the invention.

The memory subsystem may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory device such as a solid state drive, flash drive, disk drive, hard drive, subscriber identity module (SIM) card, secure digital card (SD card), micro SD card, or solid-state drive (SSD), optical and magnetic media, others, or a combination thereof.

Using the described components, the computing system 151 may be operable to produce a report and provide the report to a user and/or be controlled or programmed by a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g, a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

In certain aspects, fluid, such as lymph fluid, drained using the disclosed systems and devices is collected for downstream analysis of biomarkers. Non-limiting examples analyzing biomarkers include nucleic acid sequencing, PCR, quantitative PCR, digital droplet PCR, Western blot target capture, proteomics, nucleic acid expression analysis, antibody screening, and the like. In certain aspects, the disclosed methods obtain at least one sequencing or molecular measure of a subject's condition, including but not limited to, residual cancer, infection, immune environment, transplant rejection, and risk of poor wound healing.

In certain aspects, systems of the invention may be used for the collection, preservation, and quantification of tumor-associated cells or nucleic acids/cfDNA as measures of residual cancer, bacteria or virus-derived nucleic acids as early measures of wound infection, and transplant-derived nucleic acids as measures of transplant rejection. By way of an additional non-limiting example, the surgical drainage system disclosed herein may be used, in combination with the analysis methods described above, may be used for the collection and preservation of immune cells or immune-derived nucleic acids as measures of systemic or tumor immunity.

In various aspects, the disclosed surgical drainage system is suitable for use as a surgical drain for a variety of surgical sites and surgery types including, but not limited to, breast cancer resection and lymph node dissection, neck dissection, thoracic surgery for lung cancer with chest tube drain placement, abdominal surgery, colorectal cancer resection, pancreatic cancer resection (Whipple procedure), gynecologic cancer surgery, or prostate surgery requiring drain placement.

In various aspects, the surgical drainage system may be used in place of any suction-based surgical drainage device without limitation. In some aspects, the surgical drainage system may be adapted for use as a wound vac device configured for insertion into wounds.

The lymphatic or lymph system is central to the body's immune system. The system contains lymphatic vessels that collect lymphatic fluid from peripheral tissues of the body and transport it to lymphatic ducts. Lymph nodes of the system contain immune cells to fight infections and filter extra-cellular materials (e.g., cellular debris and byproducts, damaged cells, cancer cells, and pathogens) from the lymph fluid. The right lymphatic duct and thoracic duct drain lymph fluid collected from the lymphatic system and return it to the bloodstream via the subclavian vain.

The lymph system is associated with many types of cancer and other pathologies. For example, cells of the lymph system itself, such as in the lymph nodes, can be the source of cancers, such as lymphomas. Moreover, the lymph system can remove cancer cells (e.g., those the break away from a tumor) or other pathogens from a peripheral tissue of the body as part of its immune system functioning. Occasionally, cancer cells attach to a portion of the immune system, including after collection, and begin to grow. This metastasis is often exacerbated as the lymph system continues to circulate newly grown cancer cells throughout the system, and by extension, the body. Thus, for example, many cancers of the head, neck, breast, and glandular systems are associated with metastasis or other changes in the lymphatic system.

Accordingly, the devices disclosed herein may be inserted after surgical interventions that involve the lymphatic system in order to drain and collect lymphatic fluid. This may include direct interventions of the lymphatic system, such as resection, dissection, or excision surgeries to remove a diseased portion of the lymphatic system or to obtain tissue samples.

The present Inventors have discovered that this discarded lymphatic fluid contains important biomarkers, which may provide critical diagnostic and prognostic information. For example, certain methods of the invention use biomarkers obtained from lymphatic fluid to provide clinically significant information of a tumor in a subject, including identifying whether it is cancerous and/or whether the cancer has metastasized or is at risk of metastasis. Moreover, given its circulating activity and distribution throughout the body, the Inventors discovered that the relative movement of biomarkers in the lymph fluid may provide diagnostic or prognostic information regarding a tumor located a known distance from the point at which the lymphatic fluid was collected.

Advantageously, because lymphatic fluid samples are collected during routine clinical practice and subsequently discarded, the collection and analysis of lymphatic fluid using the systems of the invention adds little to no risk or inconvenience to the patient or clinician while providing material that is rich with clinical value.

The present invention includes systems that drain lymphatic fluid obtained directly from lymphatic tissues (e.g., lymphatic vessels or nodes) or from fluid drained from a site proximal to an infection or surgery a subject. In preferred aspects, the drained fluid is produced by the subject in response to a surgical procedure or infection, and collected using a surgical drain inserted into the patient. In certain aspects, the lymphatic fluid is collected proximal a site of a surgical wound, such as the site of a tumor resection. Collecting lymph fluid that drains from a subject, using the systems of the invention, allows longitudinal monitoring of the subject's condition by analyzing biomarkers in the fluid at multiple time points.

Advantageously, the presently disclosed systems may be used to perform longitudinal analysis of a subject. Fluid drained from a may be collected at a number of time points. This generally occurs using a dual-port drain as disclosed herein, which remains in the patient and continually collects excess fluid, e.g., lymph fluid, from the subject. The drain may be inserted into the patient solely to collect fluid samples. However, surgical drains often accompany surgical procedures or in order to drain fluid caused by and injury or infection—time periods when assessing a subject's condition over time is of pressing concern. Advantageously, analyzing the collected fluid over time puts no more burden on the subject, as the fluid is otherwise disposed as waste. Thus, without inducing further injury on the patient, the presently disclosed systems and methods may provide longitudinal insights into a subject's recovery, the efficacy of an administered treatment, and the progression of disease.

For example, in certain aspects, the disclosed systems and methods may be used following a surgical procedure for the treatment of cancer, e.g., a tumor removal. Fluid, e.g., lymph fluid, is collected using a drain inserted after the surgery. Biomarkers in the collected fluid are used to provide an assessment of whether the tumor removal removed all cancer or if the cancer is continuing to grow and/or show signs of spreading. In response to the results of the biomarker assessment, additional or adjuvant therapies may be provided via the second port. For example, an immunotherapy compound, an antibiotic, an antiviral compound, a cancer therapy, nutrients, saline, coagulant/anti-coagulant, and/or any other therapeutic as required, e.g., to promote healing or in response to biomarkers analyzed in the collected lymphatic fluid.

Systems of the invention provide an avenue for non-invasive, postoperative disease management by evaluating by-products collected from lymphatic fluid drained from a surgery site in a subject, e.g., the site of a tumor removal. The fluid recovered using the dual-port drains disclosed herein may contain material informative of an excised tumor as well as the milieu that surrounded the tumor. Accordingly, the invention recognizes that lymphatic fluid is of high clinical interest not only because of its relevance to a subject's tumor, but also for the insight it provides into the physiological conditions that gave rise to the tumor. Biomarkers collected from lymphatic fluid near a site of an excised tumor can inform on a patient's immune and/or inflammatory response following the tumor resection. Quantities of certain biomarkers, and combinations thereof, can be correlated with known patient outcomes to determine a disease prognosis. Accordingly, systems of the invention may collect biomarkers in drained lymphatic fluid for analysis to provide information regarding residual disease or determine whether the disease is likely to recur.

In preferred aspects, the collected fluid includes lymphatic fluid, lymphovascular fluid, interstitial fluid or any combination thereof. Lymphatic fluid contains waste products, including cells, cellular debris, bacteria, protein, and nucleic acid. It is an insight of the invention that an analysis of these waste products can inform on disease. In certain aspects, components of the dual-port drained systems perform functions associated with separating lymphatic fluid, or components thereof, from drained fluid. According to aspects of the invention, the separated portion of drained fluid will contain a greater quantity of biomarkers than can be obtained from an equal volume of blood.

Systems of the invention are useful to detect an unwanted health condition by analyzing drained fluid from the first port. In response, and potentially even before clinical symptoms appear, a therapeutic treatment may be provided in the second port.

In certain aspects, fluorescent labels may be used to identify biomarkers collected using the dual-port drain systems of the invention. A fluorescent label or fluorescent probe is a molecule that is attached chemically to aid in the detection of a biomarker. Fluorescent labeling generally uses a reactive derivative of a fluorescent molecule known as a fluorophore. The fluorophore selectively binds to a specific region or functional group on the biomarker and may be attached chemically or biologically. Any known technique for fluorescent labeling may be used, for example enzymatic labeling, protein labeling, or genetic labeling. Any known fluorophore may also be used. Both the fluorophore and labelling technique may be selected and adjusted based on the biomarker to be identified. The most commonly labelled molecules are antibodies, proteins, amino acids and peptides which are then used as specific probes for detection of a particular target.

Fluorescent labelling may be used to identify and quantify a biomarker in a lymphatic fluid sample without separating the components of the fluid. In certain methods, by providing fluorescent labels directly into the fluid, a fluorescent microscopy or colorimetric assay may be used to identify and quantify the presence of the biomarker from a color change alone. For example, fluorescent labels may be applied to the fluid and the color change detected by an assay device coupled to the system.

When quantifying a biomarker, barcodes may be added to a biomarker to aid in amplification, detection, or differentiation of the biomarker. Barcodes may be added to biomarkers by “tagging” the biomarker with the barcode. Tagging may be performed using any known method for barcode addition, for example direct ligation of barcodes to one or more of the ends of a nucleic acid molecule or protein. Nucleic acid molecules may, for example, be end repaired in order to allow for direct or blunt-ended ligation of the barcodes. Barcodes may also be added to nucleic acid molecules through first or second strand synthesis, for example using capture probes or primers. First and second strand synthesis may be used in RNA analysis to generate tagged DNA molecules.

Unique molecular identifiers (UMI) are a type of barcode that may be provided to biomarkers in a sample to make each biomarker, together with its barcode, unique, or nearly unique. For example, with regard to nucleic acid molecules, this is accomplished by adding, e.g. by ligation or reverse transcription, one or more UMIs to each nucleic acid molecule such that it is unlikely that any two previously identical nucleic acid molecules, together with their UMIs, have the same sequence. By selecting an appropriate number of UMIs, every nucleic acid molecule in the sample, together with its UMI, will be unique or nearly unique. One strategy for doing so is to provide to a sample of nucleic acid molecules a number of UMIs in excess of the number of starting nucleic acid molecules in the sample. By doing so, each starting nucleic molecule will be provided with different UMIs, therefore making each molecule together with its UMIs unique.

UMIs are also advantageous in that they can be useful to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, “Counting Molecules in cell-free DNA and single cells RNA”, Karolinska Institutet, Stockholm Sweden, the contents of which are incorporated herein by reference.

For RNA or mRNA sequencing, sequencing may first include the steps of preparing a cDNA library from barcoded RNA, for example through reverse transcription, and sequencing the cDNA. cDNA sequencing may advantageously allow for the quantification of gene expression within the single cell, and can be useful to identify characteristics of the single cell to, for example, make a diagnosis, prognosis, or determine drug effectiveness.

Reverse transcription may be performed using without limitation dNTPs (mix of the nucleotides dATP, dCTP, dGTP and dTTP), buffer/s, detergent/s, or solvent/s, as required, and suitable enzyme such as polymerase or reverse transcriptase. The polymerase used may be a DNA polymerase, and may be selected from Taq DNA polymerase, Phusion polymerase (as provided by Thermo Fisher Scientific, Waltham, Mass.), or Q5 polymerase. Nucleic acid amplification reagents are commercially available, and may be purchased from, for example, New England Biolabs, Ipswich, Mass., USA. The reverse transcriptase used in the presently disclosed targeted library preparation method may be for example, maxima reverse transcriptase.

Reverse transcription may be performed by oligos that have a free, 3′ poly-T region. The 3′ portions of the cDNA capture oligos may include gene-specific sequences or oligomers, for example capture primers to reverse transcribe RNA guides comprising a capture sequence. The oligomers may be random or “not-so-random” (NSR) oligomers (NSROs), such as random hexamers or NSR hexamers. The oligos may include one or more handles such as primer binding sequences cognate to PCR primers that are used in the amplifying step or the sequences of NGS sequencing adaptors. The reverse transcription primers may include template switching oligos (TSOs), which may include poly-G sequences that hybridize to and capture poly-C segments added during reverse transcription.

Reverse transcription of non-polyadenylated RNA may comprise use of a capture sequence and a capture primer or probe. Primer sequences may comprise a binding site, for example a primer sequence that would be expected to hybridize to a complementary sequence, if present, on any nucleic acid molecule released from a cell and provide an initiation site for a reaction. The primer sequence may also be a “universal” primer sequence, i.e. a sequence that is complementary to nucleotide sequences that are very common for a particular set of nucleic acid fragments. Primer sequences may be P5 and P7 primers as provided by Illumina, Inc., San Diego, Calif. The primer sequence may also allow a capture probe to bind to a solid support.

Reverse transcription can also be useful for adding a barcode or a UMI, or both to cDNA. This process may comprise hybridizing the reverse transcription primer to the probe followed by a reverse transcription reaction. The complement of a nucleic acid when aligned need not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Nucleic acid molecules may advantageously be amplified prior to sequencing. Amplification may comprise methods for creating copies of nucleic acids by using thermal cycling to expose reactants to repeated cycles of heating and cooling, and to permit different temperature-dependent reactions (e.g. by Polymerase chain reaction (PCR). Any suitable PCR method known in the art may be used in connection with the presently described methods. Non limiting examples of PCR reactions include real-time PCR, nested PCR, multiplex PCR, quantitative PCR, or touchdown PCR.

Sequencing nucleic acid molecules may be performed by methods known in the art. For example, see, generally, Quail, et al., 2012, A tale of three next generation sequencing platforms: comparison of Ion Torrent, Pacific Biosciences and IlluminaMiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

The conventional pipeline for processing sequencing data includes generating FASTQ-format files that contain reads sequenced from a next generation sequencing platform, aligning these reads to an annotated reference genome, and quantifying expression of genes. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A dual-port drain, comprising
a first port for collection of lymphatic fluid; and
a second port for delivery of a therapeutic.

2. The dual-port drain of claim 1, wherein the second port delivers an immunotherapy compound.

3. The dual-port drain of claim 1, wherein the second port delivers an antibiotic or antiviral compound.

4. The dual-port drain of claim 1, wherein the second port delivers a cancer therapy.

5. The dual-port drain of claim 1, wherein the first port collects cellular material.

6. The dual-port drain of claim 4, wherein the cellular material is a tumor cell or cell-free tumor DNA.

7. The dual-port drain of claim 1, wherein the first port comprises a suction drain.

8. The dual-port drain of claim 4, wherein the dual-port drain comprises an assay device for detecting cellular material from the first port.

9. The dual-port drain of claim 1, wherein the first port comprises a first valve for sealing the first port from collecting the lymphatic fluid.

10. The dual-port drain of claim 9, wherein the second port comprises a second valve for sealing the second port from delivering the therapeutic.

11. The dual-port drain of claim 10, wherein the first valve and/or second valve are reversible.

12. The dual-port drain of claim 11, wherein the first valve and/or second valve act independently.

13. The dual-port drain of the claim 11, wherein when delivery of the therapeutic is finished, the second valve closes the second port and the first valve closes the first port.

14. The dual-port drain of claim 1, wherein the first port comprises a sample filter for capturing solid particles.

15. The dual-port drain of claim 14, wherein the first port further comprises a heat source for moderating the temperature of collected lymphatic fluid.
